(19) United States
(12) Patent Application Publication
(10) Pub. No.: US 2005/0043273 A1
(43) Pub. Date: Feb. 24, 2005

(54) Compositions and methods for inhibiting slit protein and glypican interactions

(76) Inventor: Richard U. Margolis, New York, NY (US)

Correspondence Address: Kenneth I. Kohn;KOHN & ASSOCIATES, PLLC, Suite 410, 30500 Northwestern Highway, Farmington Hills, MI, 48334, US

(21) Appl. No.: 10/917,991

(22) Filed: Aug. 13, 2004

Related U.S. Application Data
(60) Provisional application No. 60/494,906, filed on Aug. 13, 2003.

(51) Int. Cl. A61K 38/38; A61K 31/728; A61K 31/737; A61K 31/727; A61K 31/655

(52) U.S. Cl. 514/54; 514/56; 514/12; 514/680; 424/94.64; 514/150; 514/58; 514/59

(57) ABSTRACT

A composition for inhibiting slit protein and glypican interactions including an effective amount of a heparin mimetic. A pharmaceutical composition for inhibiting slit protein and glypican interactions including an effective amount of a heparin mimetic and a pharmaceutical carrier. A composition for promoting axonal regeneration including an effective amount of a heparin mimetic. A therapeutic composition for inhibiting slit protein and glypican interaction or promoting axonal regeneration including an effective amount of a heparin mimetic. Various methods for inhibiting slit protein and glypican interaction, promoting axonal regeneration, and treating spinal cord injury.

### CROSS REFERENCE TO RELATED APPLICATIONS

This application claims the benefit of priority under 35 U.S.C. Section 119(e) of U.S. Provisional Patent Application No. 60/494,906, filed Aug. 13, 2003, which is incorporated herein by reference in its entirety.

### BACKGROUND OF THE INVENTION 1. TECHNICAL FIELD

The present invention relates to pharmaceutical compositions and methods related to the same. Specifically, the present invention relates to compositions and methods related to axonal regeneration. 2. BACKGROUND ART

There are many known proteins existing in the nervous system that are important in the function, growth, and development of neural cells. Two such proteins are (1) proteoglycans, specifically glypican-1, and (2) Slit proteins.

Proteoglycans are composed of a central core protein carrying one or more sulfated glycosaminoglycan side chains and other types of shorter oligosaccharides (Margolis et al., 1996; Margolis and Margolis, 1997; Bandtlow and Zimmermann, 2000; Yamaguchi, 2001). The core proteins vary in size from 11,000 to greater than 200,000 Da, while the number of glycosaminoglycan chains number from 1 to more than 100. The major sulfated glycosaminoglycans in nervous tissue are chondroitin sulfate and heparan sulfate. These are high molecular weight linear carbohydrate polymers composed of disaccharide repeating units of an uronic acid (D-glucuronic acid or L-iduronic acid) and an amino sugar (N-acetylgalactosamine and N-acetylglucosamine in chondroitin sulfate and heparan sulfate, respectively). The glycosaminoglycans (with the exception of hyaluronan, which does not occur in a proteoglycan form) are generally linked via their reducing ends to hydroxy amino acid residues of a core protein, with the most common linkage sequence being -glucuronosyl-galactosyl-galactosyl-xylosyl-D-serine. Biosynthesis of the glycosaminoglycan chains proceeds by repeated alternating addition of hexosamine and uronic acid residues. Because there is no precise termination mechanism, the chains display considerable length polydispersity. After polymerization the chains are modified into their final structure by various sulfotransferases and, in the case of heparan sulfate and dermatan sulfate, an uronosyl epimerase converts some D-glucuronic acid residues to L-iduronic acid.

Heparan sulfate proteoglycans are ubiquitous components of plasma membranes and are involved in a number of biological functions such as being extracellular matrix receptors in cell-cell and cell-substrate interactions, in the organization of epithelia, in mediating the actions of fibroblast growth factor-2, and as co-receptors for extracellular matrix components such as fibronectin and the interstitial collagens (Park et al., 2000; Tumova et al., 2000; Perrimon and Bernfield, 2000; Turnbull et al., 2001). Both heparan sulfate, which is a component of proteoglycans present in many cell types, and the closely related glycosaminoglycan, heparin, which occurs in a proteoglycan produced only by mast cells, are known to exhibit a wide range of fine structural variability. These glycosaminoglycans are composed of disaccharide units containing an uronic acid (D-glucuronic acid or L-iduronic acid), a glucosamine residue, which can be either N-acetylated or N-sulfated, and sulfate, which can be present at the 2-position of iduronic acid and at the 3- and 6-positions on the N-sulfated glucosamine residues. The epimerization of D-glucuronic acid to L-iduronic acid and the deacetylation and sulfation reactions, all of which occur at the polymer level, are effected by a series of enzymatic reactions, which must proceed in a defined sequence (Lindahl et al., 1998).

It has been assumed that the large number of structures made possible by these various modifications can provide a means of conveying information of biological significance, but it has generally not been possible to assign specific functions to identified structural features. It has been established, however, that the anticoagulant activity of heparin depends on its specific binding to antithrombin, and that the antithrombin-binding site is a pentasaccharide containing a unique 3-O-sulfated glucosamine N-sulfate residue (Lindahl et al., 1998). A major fibroblast growth factor-2 binding sequence in tetradecasaccharide or hexadecasaccharide fractions of human, porcine, and mouse heparan sulfates has also been identified as a cluster of five contiguous iduronic acid 2-O-sulfate (α1-4) glucosamine N-sulfate disaccharide units (Turnbull et al., 1992; Habuchi et al., 1992).

One example of a proteoglycan is glypican-1. Glypican-1 is a member of a family of glycosylphosphatidylinositol-anchored heparan sulfate proteoglycans, which is composed of six vertebrate proteins. Glypican-1 co-localizes with Slit proteins. As described below, proteoglycans have axon-growth-inhibitory properties in vitro and are up regulated at sites of central nervous system injury.

Slit proteins are high-affinity ligands of the heparan sulfate proteoglycan glypican-1 (Liang et al., 1999; Ronca et al., 2001). These proteins regulate axonal guidance, branching, dendritic development, and neural migration (See, Brose and Tessier-Lavigne, 2000; Richards, 2002; Ba-Charvet et al., 2002; Piper and Little, 2002). Slit proteins were first identified as a secreted protein expressed by midline glia in Drosophila. Analysis of null-mutations of Slit proteins indicates that they are necessary for the proper formation of commissural and longitudinal axon tracts in the fly (Rothberg, 1990). Genetic and biochemical studies provide strong evidence that Slit proteins are ligands for the repulsive guidance transmembrane receptor Roundabout (Robo), and that it functions as a short-range chemorepellent for axons crossing the midline and as a long-range chemorepellent for the migration of axons away from the ventral midline (Kidd, 1999; Battye, 1999). Three Robo receptors (i.e., Robo-1, Robo-2 and Rig-1) and three Slit proteins (Slit-1, Slit-2 and Slit-3) have been cloned as the mammalian homologues of the Drosoghila counterparts and are found to be expressed, with complementary patterns, in various parts of the developing and adult brain and spinal cord. The mechanism of midline guidance appears to have been conserved in vertebrates, since the chemorepellent activity of Slit proteins has been demonstrated on various types of nervous tissue explants.

Slit proteins repel spinal motor neurons and hippocampal and olfactory bulb axons (Brose et al., 1999; Li et al., 1999; Ba-Charvet et al., 1999) and can participate in the repulsive activity of the septum on the neurons that migrate towards the olfactory bulb from the subventricular zone (Hu, 1999; Wu et al., 1999). The Robo/Slit receptor-ligand couple is involved in regulating the growth and branching (Ba-Charvet et al., 2001) of axons projecting to appropriate regions of the brain. Moreover, the Robo/Slit receptor-ligand couple functions as a repellent and prevents axons from crossing non-target areas.

There is considerable evidence that chondroitin sulfate and heparan sulfate proteoglycans play critical roles in cell interactions that are responsible for the normal histogenesis of the central nervous system. Aside from their role in normal developmental processes, both types of proteoglycans are involved in the pathogenesis of certain neurological disorders. Neurocan and phosphacan have potent inhibitory effects on neural cell adhesion and neurite outgrowth (Friedlander et al., 1994; Milev et al., 1994). Furthermore, neurocan, phosphacan, other chondroitin sulfate proteoglycans (e.g., brevican, versican, NG2, and the like), and extracellular matrix proteins (e.g., tenascin-C) have axon-growth-inhibitory properties in vitro and are up regulated at sites of central nervous system injury (Tang et al., 2003). The expression of heparan sulfate proteoglycans (both glypican-1 and syndecan family members) is also increased in injured brain (Iseki et al., 2002). Although glypican-1, like other proteoglycans such as neurocan, is expressed exclusively by neurons in the normal central nervous system (Karthikeyan et al., 1994; Engel et al., 1996), expression also appears in reactive astrocytes of injured brain and spinal cord.

Slit proteins, which regulate axonal guidance, branching, dendritic development, and neural migration, are high-affinity ligands of the heparan sulfate proteoglycan glypican-1 (Liang et al., 1999; Ronca et al., 2001). In view of evidence for the role of cell surface heparan sulfate in the repulsive guidance activities of Slit-2 protein and the finding of high-affinity glypican-Slit interactions, it has recently been demonstrated that both glypican-1 and Slit mRNA are strongly up-regulated and co-expressed in the reactive astrocytes of injured adult brain (Hagino et al., 2003a,b). Additionally, there are dynamic changes in glypican-1 expression in dorsal root ganglion neurons after peripheral and central axonal injury (Bloechlinger et al., 2004). Moreover, high glypican-1 expression persists until the injured axons reinnervate their peripheral targets and glypican-1 is up regulated after axonal injury, which can contribute to an altered sensitivity to axonal growth or guidance (Bloechlinger, et al., 2004). All of these studies provide evidence of a function of glypican-Slit protein complexes or proteolytic processing fragments of Slit in the adult CNS (where few axon guidance events occur) as significant components of the inhibitory environment after injury.

Glypican-1, a heparan sulfate proteoglycan, interacts with Slit proteins. The glypicans share an N-terminal signal sequence followed by a globular domain containing a characteristic pattern of 14 cysteine residues, a presumably a more extended domain with the heparan sulfate (HS) attachment sites (suggesting that the HS chains are deployed close to the cell surface), and a hydrophobic C-terminal sequence that is involved in the formation of the GPI anchor structure. Glypican-1 has a 56 kDa core protein and 3-4 heparan sulfate chains. Northern analysis has demonstrated high levels of glypican-1 mRNA in brain and skeletal-muscle, and in situ hybridization histochemistry has shown that glypican-1 mRNA is especially prominent in cerebellar granule cells, large motor neurons in the brain stem, and CA3 pyramidal cells of the hippocampus (Karthikeyan et al., 1994). As a result of the work disclosed by Karthikeyan, et al. along with results from parallel immunocytochemical studies, glypican-1 has been proven to be predominantly a neuronal product in the late embryonic and postnatal rat nervous system.

The functions of glypican-1 in nervous tissue have been linked to endogenous ligands. Proteins or ligands involved in axonal guidance can have attractant or repulsive effects. These proteins or ligands can be further subdivided into diffusible molecules, which mediate long-range effects, and cell surface or extracellular matrix proteins, which are involved in short-range attraction and repulsion. Moreover, a single molecule can have dual functions. This is the case not only for large, multi-domain extracellular matrix proteins such as tenascin-C (Prieto et al., 1992) and Slit proteins (Sang et al., 2002; Englund et al., 2002), but also for diffusible growth cone guidance molecules such as netrin-1 (Colamarino and Tessier-Lavigne, 1995). Insofar as the C-terminal portion of Slit proteins is released from the cell membrane by in vivo proteolytic processing, Slit proteins are capable of exerting both short- and long-range effects.

In studies aimed at characterizing glypican-Slit interactions in more detail, recombinant human Slit-2 protein and the N- and C-terminal portions generated by in vivo proteolytic processing was used in an ELISA to measure binding of a glypican-Fc fusion protein (Ronca et al., 2001). Saturable and reversible high-affinity binding, which did not require the presence of divalent cations was seen to the full-length protein and to the C-terminal portion that is released from the cell membrane, with dissociation constants in the 80-110 nM range, whereas only a relatively low level of binding was detected to the larger N-terminal segment.

Co-transfection of 293 cells with Slit proteins and glypican-1 cDNAs followed by immunoprecipitation demonstrates that glypican-Slit interactions occur in vivo. The binding affinity of the glypican core protein to Slit is an order of magnitude lower than that of the glycanated protein, and the O-sulfate groups on the heparan sulfate chains play a critical role in the interactions of glypican-1 with Slit proteins. Analysis of deletion mutants of the C-terminal portion of Slit-2 demonstrates that most of the glypican binding can be ascribed to the first EGF-like repeat and the adjacent 178-amino acid ALPS domain (also found in agrin, laminin and perlecan). These findings demonstrate that glypican binding to the releasable C-terminal portion of Slit proteins serves as a mechanism for regulating the biological activity of Slit proteins and/or the proteoglycan, and acquire additional significance from studies demonstrating a role of cell surface heparan sulfate in the repulsive guidance activities of Slit-2 protein (Hu, 2001).

Glypican-1 contains a nuclear localization signal, is present in the nuclei of central nervous system neurons, and is transported to the nuclei of 293, COS-1, and C6 glioma cells, which show changes in the pattern of glypican nuclear immunoreactivity both during cell division and correlated with different phases of the cell cycle (Liang et al., 1997). These findings suggest that glypican-1 can be involved in the regulation of cell division and survival by directly participating in nuclear processes. Two nuclear export signal sequences, which function via a leptomycin B-sensitive, CRM1-mediated export mechanism has been identified. Using an affinity matrix in which a recombinant glypican-Fc fusion protein expressed in 293 cells was coupled to protein A-Sepharose, two rat brain proteins were detected and isolated by SDS-PAGE as a single 200 kDa silver-stained band, from which 16 partial peptide sequences were obtained by nano-electrospray tandem mass spectrometry (Liang et al., 1999). Mouse expressed sequence tags containing two of these peptides were employed for oligonucleotide design and synthesis of probes by PCA, and enabled isolation from a rat brain cDNA library a 4.1 kb clone that encoded two of the peptide sequences and represented the N-terminal portion of a protein containing a signal peptide and three leucine-rich repeats. Comparisons with recently published sequences showed that these peptides were derived from proteins that are members of the Slit protein family, which share a number of structural features such as N-terminal leucine-rich repeats and C-terminal epidermal growth factor-like motifs.

All of the five known rat and human Slit proteins contain 1523-1534 amino acids, and the isolated peptide sequences corresponded best to those present in human Slit-1 and Slit-2. Northern analysis demonstrated the presence of two mRNA species of 8.6 and 7.5 kb using probes based on both N- and C-terminal sequences, and in situ hybridization histochemistry showed that these glypican-1 ligands are synthesized by neurons, such as hippocampal pyramidal cells and cerebellar granule cells, where it was previously demonstrated as having glypican-1 RNA and immunoreactivity.

As set forth above, there is evidence for the role of cell surface heparan sulfate in the repulsive guidance activities of Slit-2 protein (Hu, 2001) and Slit protein mRNA is strongly up-regulated and co-expressed with glypican-1 mRNA in the reactive astrocytes of injured adult brain (Hagino et al., 2003). Accordingly, there is a need for compositions that interfere with the interaction between glypican-1 and Slit proteins in order to prevent and/or reverse damage resulting from spinal cord injury.

### SUMMARY OF THE INVENTION

The present invention provides a composition for inhibiting slit protein and glypican interactions including an effective amount of a heparin mimetic. Additionally, the present invention provides a pharmaceutical composition for inhibiting slit protein and glypican interactions including an effective amount of a heparin mimetic and a pharmaceutical carrier. The present invention further provides a composition for promoting axonal regeneration including an effective amount of a heparin mimetic. Furthermore, the present invention provides a therapeutic composition for inhibiting slit protein and glypican interaction or promoting axonal regeneration including an effective amount of a heparin mimetic. Finally, the present invention provides various methods for inhibiting slit protein and glypican interaction, promoting axonal regeneration, and treating spinal cord injury.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 illustrates the inhibition of glypican-1 binding to Slit proteins as a function of size of structurally defined heparin oligosaccharides.

### DETAILED DESCRIPTION OF THE INVENTION

Generally, present invention utilizes compositions that affect and/or inhibit glypican and Slit protein interactions. The present invention can be used for promoting axonal regeneration and treatment of spinal cord injury occurring in any type of animal and human being.

The present invention provides a composition and method for inhibiting glypican and Slit protein interactions, which results in promoting axonal regeneration. The present invention also provides a composition and method for repairing and/or preventing of paralysis following spinal cord injury. The present invention further provides a mechanism of repairing and/or preventing paralysis by inhibiting glypican and Slit protein interactions. Furthermore, the present invention provides a composition, mechanism, and method of affecting axonal regeneration and guidance, increasing axonal branching, increasing and/or improving dendritic development, and fostering neural cell migration.

Slit proteins regulate axonal guidance, branching, dendritic development, and neural migration, are high-affinity ligands of the heparan sulfate proteoglycan glypican-1 (Liang et al., 1999; Ronca et al., 2001). In view of evidence for the role of cell surface heparan sulfate in the repulsive guidance activities of Slit-2 protein and the finding of high-affinity glypican-Slit interactions, it has recently been shown that both glypican-1 and Slit mRNA are strongly up-regulated and co-expressed in the reactive astrocytes of injured adult brain (Hagino et al., 2003a,b), and that there are dynamic changes in glypican-1 expression in dorsal root ganglion neurons after peripheral and central axonal injury (Bloechlinger et al., 2004). Because the smaller C-terminal proteolytic processing product of Slit binds with high affinity to glypican-1, this also prevents its diffusion from sites of central nervous system injury. Whether any adverse effects on axonal regeneration are due to a glypican-Slit complex or the retention of C-terminal Slit protein fragments at the injury site, it is possible that drugs with heparin-like activity can limit the functional consequences of spinal cord injury. Further, because the blood-brain barrier breaks down at sites of central nervous system injury, this does not represent a significant impediment to drug penetration after systemic administration. The time-course of up-regulation of Slit after injury is also therapeutically favorable, insofar as the expression of Slit-2 mRNA peaks at one week and becomes undetectable by two weeks post-injury (Hagino et at., 2003).

As set forth above, proteoglycans have axon-growth-inhibitory properties and are up-regulated at sites of central nervous system injury. Cell surface heparan sulfate can be involved in the repulsive guidance activities of Slit proteins. Further, based on findings of high affinity glypican-Slit interactions, it has been shown that Slit mRNA is strongly up-regulated and co-expressed with glypican-1 mRNA in the reactive astrocytes of injured adult brain. This demonstrates a function of Slit proteins and glypican-1 in the adult CNS (where few axon guidance events occur) as significant components of the inhibitory environment after injury. Thus, use of compounds that interfere with Slit protein and glypican interactions can have a strongly beneficial effect on limiting, or promoting recovery from, the functional consequences of spinal cord injury. Such compounds are those that have heparin-like activity.

The term “effective amount,” as used herein, means, but is not limited to, the amount determined by such considerations as are known in the art of treating or affecting the described glypican-Slit protein interactions, wherein it must be effective to provide measurable relief in treated individuals such as exhibiting improvements including, but not limited to, improved movement, more rapid recovery, improvement or elimination of symptoms or reduction of complications, promoted axonal regeneration, affected axonal guidance, increased cell growth, increased axonal branching, or other measurements as appropriate and known to those skilled in the medical arts.

The term “heparin mimetic(s),” as used herein, means, but is not limited to, a compound, composition, or molecule that can interfere with glypican-Slit protein interactions. Basically, any type of compound, composition, or molecule that has the capability of affecting, interfering, and/or inhibiting glypican-Slit protein interactions can be a heparin mimetic. Additionally, the heparin mimetic can have complete, negligible, or incomplete anticoagulant activity. Preferably, the heparin mimetic is a small molecule that contains essential functional groups, often with additional hydrophobic or charged groups, to resemble the active conformation of the parent heparin structure. There are numerous types of heparin mimetics or carbohydrate mimetics known to those of skill in the art and are further exemplified below.

The basis of the present invention is a mechanism that affects and/or interferes with glypican and Slit protein interactions. Specifically, the mechanism is a heparin mimetic or any composition that can affect and/or interfere with glypican and Slit protein interactions. There are numerous compositions that can affect and/or inhibit protein-protein interactions. (See, Gadek and Nicholas, 2003, which are incorporated by reference in its entirety). Although the application of agents that affect protein-carbohydrate interactions is even more recent, good progress has been made in understanding the supramolecular chemistry of carbohydrate recognition by receptors through noncovalent interactions, and in combinatorial chemistry using glycopeptide and oligosaccharide libraries (Davis and Wareham, 1999; St. Hilaire and Meldal, 2000). The development of carbohydrate-based drugs has been slowed because glycosidases in the blood can reduce their half-life to just a few minutes, depending on the structure. Carbohydrate-based drugs such as heparin have avoided this pharmacokinetic pitfall because their structures are not readily recognized by the body's normal complement of glycosidases. Moreover, recent advances have demonstrated that these problems can be circumvented with the use of carbohydrate or heparin mimetics.

The use of “heparin mimetics” for the present invention is well-suited for affecting and/or interfering with glypican-Slit protein interactions. First, the blood-brain barrier breaks down at sites of central nervous system injury and thus does not represent a significant impediment to drug penetration after systemic administration. Second, the time-course of up-regulation of Slit proteins after injury is therapeutically favorable, insofar as the expression of Slit-2 mRNA peaks at one week and becomes undetectable by two weeks post-injury (Hagino et at., 2003). This indicates the possibility of a relatively short duration of therapy during an early “critical period,” which minimizes any drug toxicity and allow the use of higher doses of systemically administered drugs if necessary. It also provides the option of local administration (e.g., intrathecally by catheter or from an implanted depot) to achieve higher drug concentrations at the site of injury.

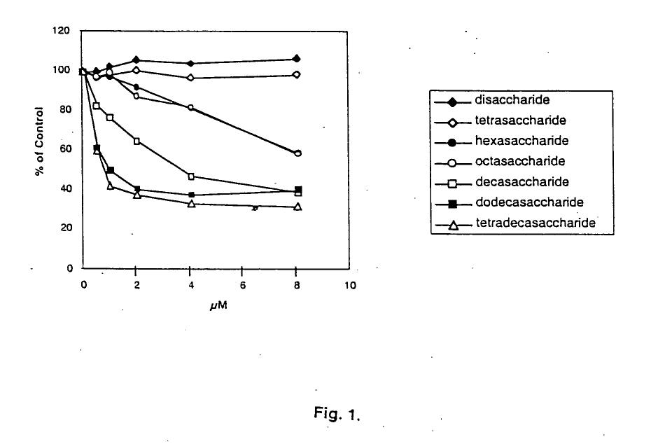

As set forth in the Background Art section, glypican-1 containing heparan sulfate chains bind to Slit proteins with an affinity that is an order of magnitude greater than that of the glypican core protein, and O-sulfate groups on the heparan sulfate are critical for this binding (Ronca et al., 2001). A report by Hu (2001) has also emphasized the importance of cell surface heparan sulfate in the repulsive guidance activities of Slit-2 protein. Using ELISA, the number of heparan sulfate disaccharide units that participate in binding to Slit proteins has been determined by inhibition studies utilizing heparin oligosaccharides of defined structure (Pervin et al., 1995). These studies demonstrate that the maximum inhibition of glypican binding begins to be seen with a heparin decasaccharide and is more extensive with the dodecassacharides and tetradecasaccharides (i.e., 6-7 disaccharide units; See, FIG. 1).

In any embodiment of the present invention, a composition is utilized that interferes with glypican and Slit protein interactions. The composition can be a heparin mimetic. Heparin mimetics include, but are not limited to, chemically modified glycosaminoglycans such as hyaluronic acid, dermatan sulfate, chondroitin sulfate, heparin, heparan sulfate, and keratan sulfate, low molecule weight heparin-mimetic compounds, heparin oligosaccharides, heparin-like glycosaminoglycans (HLGAGs), suramin, suramin-like compounds, polyanions such as dextran sulfate, sulfated polysaccharides, negatively charged serum albumin and milk proteins, synthetic sulfated polymers, polymerized anionic surfactants and polyphosphates, various sulfated molecules, various sulfonated molecules, synthetic polyaromatic compounds, polyaromatic compounds synthesized by polymerization of aromatic ring monomers with formaldehyde (which yields substantially ordered backbones with different functional anionic groups (hydroxyl and carboxyl) on the phenol ring), polysulfated dyes (including, but not limited to, Reactive Black 5, Remazol Brilliant Blue R, Reactive Orange 16, trypan blue), α-cyclodextrin sulfate (Aldrich/Fluka), fully sulfated maltotrioside prepared as a precursor for synthetic heparins (Petitou et al., 1999), water-soluble synthetic dextran derivatives such as those containing sulfate, carboxymethyl, and benzylamide groups on the OH residues of glucose units (Ledoux et al., 2003), randomly derivatized dextrans (e.g., derivatized with benzylamidesulfonate, carboxymethyl, etc.)(Tardieu et al., 1992), PI-88 (a sulfated phosphomannan) (Progen Industries Ltd., Brisbane; Yu et al., 2002), heparin-derived oligosaccharide C3 (Ma et al., 2002), GL-522-Y-1 (which is a cyclic octaphenol-octasulfonic acid), a low-molecular weight fragment of heparin prepared by chemical or enzymatic depolymerization and that is preferably devoid of anticoagulant activity, fragments thereof, combinations thereof, and any other similar compositions capable of affecting and/or inhibiting glypican and Slit protein interactions known to those of skill in the art. Specifically referring to these heparin mimetics, for example, heparin oligosaccharides inhibit glypican-Slit interactions and can more easily penetrate to sites of CNS injury, while having no significant anticoagulant activity. Another example of a heparin mimetic is a relatively small, sulfated molecule such as the anti-parasitic drug suramin, which also inhibits glypican-Slit interactions to the same extent as large heparin oligosaccharides.

In any embodiment of the present invention, the heparin mimetic of composition can be chemically or enzymatically modified to diminish and/or neutralize any anticoagulant activity of the heparin mimetic. Such chemical or enzymatic modification is well known to those of skill in the art. Alternatively, the composition of the present invention can further include another component that diminishes and/or neutralizes any anticoagulant activity of the heparin mimetic. Such secondary compositions include, but are not limited to, platelet factor IV, prothrombin, vitamin K, fibrinogen, prothrombin, thromboplastin, tissue factor, calcium, labile factor, stable factor, antihemophilic globulin (AHF), antihemophilic globulin (AHG), antihemophilic factor A, plasma thromboplastin component, Christmas factor, antihemophilic factor B, Stuart factor, Prower factor, Stuart-Prower factor, plasma thromboplastin antecedent (PTA), antihemophilic factor C, Hageman factor, surface factor, contact factor, fibrin stabilizing factor (FSF), fibrin stabilizing enzyme, fibrinase, prekallikrein (Fletcher factor), high molecular weight kininogen (Fitzgerald), other blood clotting or coagulation factors, combinations thereof, and any other similar compound that can inhibit the anti-coagulant activity of the heparin mimetic compound known to those of skill in the art.

In addition to the composition, the present invention provides for various pharmaceutical compositions including the heparin mimetic and a suitable pharmaceutical composition. Further, the present invention provides for a therapeutic composition for preventing slit protein and glypican interactions or for promoting axonal regeneration. This therapeutic composition includes the heparin mimetic of the present invention.

In another embodiment, the present invention provides a method of inhibiting slit protein and glypican interactions by administering an effective amount of the composition of the present invention. A further embodiment provides a method of promoting axonal regeneration by administering an effective amount of the composition of the present invention. Finally, the present invention provides for a method of treating and/or preventing spinal cord injury by administering an effective amount of the composition of the present invention.

The compositions and methods described herein are administered according to known pharmaceutical methods and techniques. The composition of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art and as described herein.

The composition made in accordance with the present invention can be prepared and administered in a wide variety of dosage forms. For example, these pharmaceutical compositions can be made in inert, pharmaceutically acceptable carriers that are either solid or liquid. Solid form preparations include powders, tablets, dispersible granules, capsules, cachets, injections, and suppositories. Other solid and liquid form preparations could be made in accordance with known methods of the art. The quantity of active composition in a unit dose of preparation can be varied or adjusted from 1 mg to about 300 mg/kg (milligram per kilogram) daily, based on an average 70 kg patient. The dosages, however, can be varied depending upon the requirement with a patient, the severity of the condition being treated, and the composition being employed. Determination of the proper dosage for particular situations is within the skill of the art.

The composition of the present invention can be administered intrathecally. Intrathecal administration is advantageous because this route largely bypasses the blood-brain barrier. Further, by providing a high local concentration of the composition, toxicity can be reduced or eliminated, which could result from systemic administration in high enough doses to achieve the required concentration at the spinal cord injury site. Intrathecal administration can occur by any manner known by those of skill in the art. For example, intrathecal delivery can occur through an implanted depot of collagen (Hamann, et al., 2003) or other biocompatible, biodegradable, injectable, and fast gelling biomaterial (e.g., hyaluronan) known to those of skill in the art. Such implanted material provide for higher drug concentrations at the site of injury. A more specific example of a hyaluronan is a high molecular weight divinylsulfone cross-lined hyaluronan preparation. The degree of crosslinking of this hyaluronan preparation is about 1/20 monosaccharide residues, and at equilibrium hydration it has a polysaccharide concentration of ˜0.5%. Although it appears to be a solid gel, the actual slurry of gel particles is very plastic (e.g., can be extruded through a 30 gauge needle) and can stay in place for adequate periods (days to weeks).

In the method of the present invention, the composition of the present invention can be administered in various ways. The composition can be administered as the compound, a pre-cursor, or as pharmaceutically acceptable salt. For example, the composition of the present invention can be an inactive pre-cursor composition from which the active drug is generated in vivo by enzymatic or other activities. The composition of the present invention can be administered alone or as an active ingredient in combination with pharmaceutically acceptable carriers, diluents, adjuvants and vehicles. The compositions can be administered orally, subcutaneously or parenterally including intravenous, intraarterial, intramuscular, intraperitoneally, and intranasal administration as well as intrathecal and infusion techniques. Implants of the compositions are also useful. For example, implants can be a depot of collagen (Hamann, et al., 2003) or other biocompatible, biodegradable, injectable, and fast gelling biomaterial (e.g., hyaluronan) known to those of skill in the art. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans can be treated longer than the mice or other experimental animals exemplified herein, which treatment has a length proportional to the length of the disease process and drug effectiveness. The doses can be single doses or multiple doses over a period of several days, but single doses are preferred. The treatment generally has a length proportional to the length of the disease process and drug effectiveness and the patient species being treated.

When administering the composition of the present invention parenterally, it can generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it can be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used is compatible with the composition of the present invention.

Sterile injectable solutions can be prepared by incorporating the compositions utilized in practicing the present invention in the required amount of the appropriate solvent with several other ingredients, as desired.

A pharmacological formulation of the present invention can be administered to the patient in an injectable formulation containing any compatible carrier, such as various vehicle, adjuvants, additives, and diluents; or the compositions utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the composition utilized with the present invention can be administered orally to the patient. Conventional methods such as administering the compositions in tablets, suspensions, solutions, emulsions, capsules, powders, syrups and the like are usable. Known techniques, which deliver it orally or intravenously and retain the biological activity, are preferred.

In one embodiment, the composition of the present invention can be administered initially by intravenous injection to bring blood levels to a suitable level. The patient's levels are then maintained by an oral dosage form, although other forms of administration, dependent upon the patient's condition and as indicated above, can be used. The quantity to be administered can vary for the patient being treated and can vary from about 100 ng/kg of body weight to 100 mg/kg of body weight per day and preferably can be from 1 mg/kg to 10 mg/kg per day.

The above discussion provides a factual basis for the use of the present invention described herein. The methods used with a utility of the present invention can be shown by the following non-limiting examples and accompanying figures.

### EXAMPLES

### Materials and Methods for Examples

General Methods in Molecular Biology:

Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with Flow Cytometry can be used for detection of cells containing specific DNA and mRNA sequences (Testoni et al, 1996, Blood 87:3822.)

### Example One

Preparation of Glypican-1-Fc and Human Slit-fusion Proteins.

Human embryonic kidney 293 cells were transfected with a glypican-1-Fc fusion protein construct using Lipofectamine 2000 and grown in serum-free DMEM containing 1% ITS+. To separate the glycanated form of the proteoglycan (which was used for all studies) from unglycanated core protein, the conditioned medium was applied to a 0.9×8 cm column of DEAE-Sephacel equilibrated with 150 mM NaCl, 50 mM Tris-HCl, pH 8.0. After elution with 50 mM Tris-HCl (pH 8.0) containing 0.6 M NaCl, the glycanated glypican-1-Fc was bound to protein A-Sepharose beads, eluted with 0.1 M glycine, pH 3.0, and immediately neutralized with 1 M Tris, pH 8.0, for storage at −80° C.

293 cells were transfected with the pSecTagB vector (Invitrogen, Carlsbad, Calif.) containing cDNA for the His-tagged uncleavable variant of human full-length Slit-2, in which the nine amino acids encompassing the proteolytic processing site were deleted, producing an uncleavable full-length protein. 1 M NaCl extracts of the 293 cells were incubated with nickel-agarose beads (Qiagen) for 2 hours at 4° C., and after washing, bound protein was eluted with 10 mM Hepes (pH 7.5) containing 250 mM imidazole and 1 M NaCl. Protein concentrations were determined by the Bradford assay. Purity of the recombinant Slit protein was confirmed by SDS-PAGE followed by silver staining.

ELISA assay:

96-well plates (Corning Costar #9018) were coated overnight with the human full-length Slit-2 in imadazole/NaCl elution buffer at a saturating concentration of 1-5 μg/well, after dilution in PBS. Unbound protein was removed by washing with TBST (10 mM Tris-HCl, pH 7.4, 150 mM NaCl, 0.1% Tween 20), the wells were blocked with 10% FBS in TBST for 2 hours, and then incubated for 18 hours at room temperature with glypican-1-Fc (1 μg/well) in 5 mM PBS (pH 7.2), or in PBS containing varying concentrations of potential inhibitory compositions. Bound glypican was detected using a biotinylated anti-human Fc antibody (Jackson Immunoresearch; 1:250,000 in TBST, for 2 hours), followed by incubation for 20 minutes with HRP-conjugated streptavidin (1:20,000 in TBST). The colorimetric reaction product from the o-phenylenediamine substrate was measured at 450 nm using a Dynatech MRX ELISA plate reader. Nonspecific binding was calculated as the binding of glypican-1-Fc to wells coated with BSA (2 mg/ml solution). Percent inhibition was calculated in relation to parallel assay wells in which no inhibitor was added to the glypican-1 solution. From serial dilutions of a known concentration of glypican-1-Fc directly coated on the wells and the corresponding immunoreactivity absorbance, a standard curve could also be created and used to quantitate the absolute amount of glypican-1-Fc bound.

### Example Two

Surface Plasmon Resonance.

SPR is a two-phase kinetic measurement of interaction, which is performed by immobilizing an albumin conjugate of heparin or related molecules to carboxymethylated dextran on a sensor chip and flowing a solution of Slit protein over this surface. This approach is preferable to immobilizing Slit protein because its binding site(s) might be affected by its direct chemical coupling to the sensor chip. The kinetic parameters ka (on-rate) and kd (off-rate) are evaluated using the Biosensor BIA Evaluation software according to the manufacturer's methods, and the dissociation constant, Kd, is obtained from the ratio kd/ka. The ability of SPR methodology to directly and quantitatively measure the affinities for Slit of various chemically defined heparins, heparan sulfates, and heparin oligosaccharides considerably extends the range of information on this important topic beyond that which can be obtained from inhibition studies using the ELISA assay.

Preparation of Heparin Biochip.

Preparation of the heparin biochips utilized two batches of albumin-heparin conjugate. Covalently bound conjugate of albumin-heparin relied on the condensation reaction of albumin and heparin using N-ethyl-N-(dimethyaminopropyl) carbodiimide (EDC). Unreacted albumin and heparin were removed by diethylaminoethyl (DEAE)-cellulose and Cibacron blue Sepharose chromatography, respectively. The biochip with immobilized heparin-BSA conjugate afforded a higher RU (response units). Therefore, competition studies with this chip can be performed.

The heparin biochip was prepared. First, the albumin-heparin conjugate was covalently immobilized to the biosensor surface through its primary amino groups on a C1 chip (Biosensor AB, Uppsala, Sweden). Briefly, carboxymethyl groups on the C1 chip surface were first activated using an injection pulse of 50 μl (flow rate, 5 μl/min) of an equimolar mixture of NHS/EDC (final concentration 0.05 M, mixed immediately prior to injection). Then, an albumin-heparin solution (200 μg/ml in sodium acetate buffer with 2 M guanidine hydrochloride, pH 4.0) was applied to the chip surface. Excess unreacted sites on the sensor surface were blocked with a 40 μl injection of 1 M ethanolamine. In order to confirm successful immobilization, observation of an ˜300 RU response increase was determined. To prepare the control flow cell, bovine serum albumin was immobilized on the surface using a similar coupling procedure.

SPR experiments were performed on the BIAcore 3000 (Biosensor AB, Uppsala, Sweden) apparatus operated using BIAcore 3000 version software. The buffers used in SPR were filtered and degassed. Further, kinetic measurements of heparin and Slit protein interaction were taken using SPR. For the kinetic studies of Slit interactions with heparin, measurements were performed on a BIAcore 3000. Different concentrations (50, 100, 200, 300 and 500 nM) of Slit protein in buffer (1 mM sodium phosphate, pH 7) were injected over both the albumin-heparin (Sigma) and control albumin surfaces simultaneously at a flow rate of 10 μl/min. At the end of each sample injection, the same buffer was flowed over the sensor surface to facilitate dissociation studies. After a three minute dissociation time, the sensor surface was regenerated by injection of 20 μl of 2 M NaCl. The response was monitored as a function of time (sensorgram) at 25° C. The control cell was used to subtract the contribution of non-specific interactions with the immobilized albumin on the surface. Kinetic parameters were evaluated using the BIA Evaluation software (Version. 3.1, 1999).

A solution competition study was performed between Slit protein and heparin, LMW heparin and heparin-derived oligosaccharides, utilizing SPR. Slit protein (80 nM) mixed with different concentrations of heparin, LMW heparin, disaccharide, tetrasaccharide, hexasaccharide and octasaccharide in HBS-EP buffer (0.01 M HEPES, 0.15 M NaCl, 3 mM EDTA, 0.005% polysorbate, pH 7.4) (BIAcore) were injected over both the albumin-heparin (prepared) and the control albumin surfaces at a flow rate of 25 μl/min. After each run, the dissociation and regeneration were performed as described above. For each set of competition experiments using SPR, a control experiment (only Slit protein without any heparin or oligosaccharides) was performed in order to verify that the surface was completely regenerated and that the results obtained between runs were comparable.

Kinetic Measurement of Slit Protein Interaction with Heparin.

The structure of heparin is very similar to the sulfated regions of heparan sulfate, and it has therefore been used as an excellent molecular model for heparan sulfate-protein interaction studies. These experiments require the immobilization of either heparin or a heparin-binding protein on the surface of a biosensor chip, over which its binding partner, a heparin-binding protein (or heparin) is passed. In natural biological systems, heparan sulfate is immobilized on the cell surface through its core protein and captures heparin-binding proteins that flow over the cell surface. SPR was used to obtain information concerning the kinetics of binding of heparin to Slit and its inhibition by heparin and heparin oligosaccharides. This approach can also be helpful in establishing both the minimum and optimal size of the binding domain in heparin for Slit.

Kinetic analysis of the interaction between Slit protein and heparin afforded a k_{d} value of 1.1×10⁻³ s⁻¹, a kₐ value of 3.3×10³ M⁻¹s⁻¹ and a K_{D} of 3.3×10⁻⁷ M. These binding kinetics data are comparable to previous studies on glypican-Slit interactions, which indicated a single class of high affinity binding sites with dissociation constants of 80-100 nM for full-length Slit and its C-terminal portion, and 300 nM for the N-terminal construct. It has also recently been shown that glypican-1 is a high-affinity ligand (K_{d}=10 nM) of the epidermal growth factor-related peptide, Cripto-1, which activates the tyrosine kinase c-Src as a result of this specific interaction.

A solution competition study between Slit and heparin, LMW heparin, and heparin-derived oligosaccharides using SPR was performed. To examine the effect of saccharide chain size of heparin on the Slit protein interaction, solution/surface competition experiments were performed by SPR. In each competition experiment, different amounts of heparin, LMW heparin and heparin-derived saccharide (from di- to octa-) of defined structures were added in the analyte (Slit protein) solution. When different concentrations of heparin disaccharide were present in the Slit protein/heparin interaction solution, no competition effect was observed. In all other cases, increasing concentrations of competing analytes (heparin, LMW heparin, tetrasaccharide, hexasaccaride and octasaccharide), decreased the observed binding of Slit protein. For example, when the concentration of octasaccharide was 100 μM, the interaction decreased to approximately 50% of the control value (no competing analyte present). At a concentration of octasaccharide of 500 μM, virtually no binding was observed. The IC₅₀ value is commonly defined as the concentration of competing analyte resulting in 50% of the response observed in the absence of competing analyte. The variation in IC₅₀ values observed demonstrates that the interaction between Slit protein and heparin is chain-length dependent, and that the minimum heparin oligosaccharide size that competes with heparin binding to Slit is a tetrasaccharide.

The studies described hereinabove have provided additional information on the fine structural features required for the heparan sulfate-mediated binding of glypican-1 to Slit proteins, and also demonstrated that significant inhibition of these interactions can be obtained by small sulfated molecules whose structures are entirely unrelated to those of heparin and heparan sulfate. In addition to earlier evidence for the role of cell surface heparan sulfate in the repulsive guidance activities of Slit-2 protein, it has recently been reported that both Slit-2 and glypican-1 mRNA are strongly up-regulated and co-expressed in the reactive astrocytes of injured adult brain, suggesting a possible function of Slit proteins and glypican-1 in the adult CNS (where few axon guidance events occur) as significant components of the inhibitory environment after injury. Therefore, glypican-1 and Slit proteins, either acting alone or as a complex, are a significant factor in preventing axonal regeneration after spinal cord injury. Although significant amounts of full-length unprocessed Slit are present in nervous tissue (accounting for its original identification as a glypican-1 ligand in the form of the 200 kDa protein), because the smaller C-terminal proteolytic processing product binds with high affinity to glypican-1, this would prevent its diffusion from sites of central nervous system injury. Whether any adverse effects on axonal regeneration are due to a glypican-Slit complex or the retention of C-terminal Slit protein fragments at the injury site, inhibiting their interaction with heparin-like compounds can limit the functional consequences of spinal cord injury.

### Example Three

The glypican-1-Fc and human Slit-2 fusion proteins are prepared as set forth in Example One. The following compositions have shown inhibition of slit proteins and glypican interactions:
1) Arixtra (Organon/Sanofi) octasulfated HS pentasaccharide, with an average MW of 1728 shows no effect @ 50 μM.
2) Enoxaparin (Lovenox/Aventis) with an average MW of 4,500 shows 49% inhibition @ 0.5 μM, 73% inhibition at 1 μM, 87% inhibition @ 5 μM, and 87% inhibition @ 50 μM.
3) Dalteparin (Fragmin/Pharmacia-Pfizer) with an average MW of 5,000 shows 61% inhibition @ 0.5 μM, 81% inhibition @ 1 μM, 86% inhibition @ 5 μM, and 87% inhibition @ 50 μM.
4) Heparin-derived oligosaccharide C3 (Ma et al., 2002) shows 22% inhibition @ 5 μM and 54% inhibition @ 50 μM.
5) YGD04a (a mixture of sulfated hexasaccharides) with a MW of fully-sulfated saccharide [19 sulfates] of 2942 shows 51% inhibition @ 5 μM and 64% inhibition @ 50 μM.

6) PI-88, a sulfated phosphomannan (Progen Industries Ltd., Brisbane; Yu et al., 2002) shows inhibition as follows:

| | |
|---|---|
| Peak I (˜3000 MW): | 79% inhibition @ 0.5 μM, 84% inhibition @ |
| | 5 μM, |
| | 87% inhibition @ 50 μM |
| Peak II (˜2700 MW): | 10% inhibition @ 0.01 μM, 36% inhibition @ |
| | 0.05 μM, |
| | 48% inhibition @ 0.1 μM, 62-77% inhibition @ |
| | 0.5 μM, 84% inhibition @ 5 μM, |
| | 85% inhibition @ 50 μM. |

7) GL-522-Y-1, a cyclic octaphenol-octasulfonic acid that has heparin-like antithrombotic activity on vascular endothelial cells but no significant anticoagulant activity in whole blood (Pinhal et al., 2001) shows 58% inhibition @ 1 μM, 61% inhibition @ 5 μM (vs. 29% inhibition for methylated derivative), and 67% inhibition @ 50 μM (vs. 50% inhibition for methylated).

8) A series of synthetic polyaromatic compositions synthesized by polymerization of aromatic ring monomers with formaldehyde, yielding substantially orderedbackbones with different functional anionic groups (hydroxyl and carboxyl) on the phenol ring, which have demonstrated heparin-like activity in several functional assays (Benezra et al., 2002) shows the following inhibition:

TABLE ONE

| | % Inhibition | | | |
|---|---|---|---|---|
| SAMPLE | 50 μM | 5 μM | 1 μM | Mol. Wt. |
| RG-13528-W | 10 | 4.5 | — | 100 |
| RG-14444 | — | 79 | 79 | 30,000 |
| RG-13530-W | 80 | 60 | 41 | 1,100 |
| RG-13519-W | 64 | 32 | — | 1,100 |
| RG-13524-W | 72 | 62 | 52 | 1,100 |

9) A series of sulfated dextran derivatives were tested and show the following inhibitions:

TABLE TWO
Sulfated T-40 Dextran derivatives (From Denis Barritault, Paris)

| | % Inhibition | | | |
|---|---|---|---|---|
| | 1 mg/ml | 100 μg/ml | 25 μg/ml | 2 μg/ml |
| Dextran | | | | |
| D120* | 81 | 79 | 83 | 57 |
| CR17 | 77 | 50 | 70 | 43 |
| CR21 | 79 | 73 | 80 | 46 |
| RG94 | 80 | 75 | 77 | 42 |
| DAC | 87 | 78 | 85 | 43 |
| CR27 | 86 | 77 | 83 | 56 |
| CR29 | 87 | 76 | 83 | 41 |
| CR32 | 78 | 75 | 83 | 31 |
| CR36 | 72 | 64 | 79 | 66 |
| CR34 | 3 | | | 4 |
| CR35 | 6 | | | 3 |

| | |
|---|---|
| Sulfated hydrophilic | |
| dextrans: | |
| D120 | HM-Hi (80 kDa) equivalent to RG1503 |
| CR-17 | HM-Hi (400 kDa) |
| CR-21 | HM-Hi (3500 kDa) |
| Sulfated hydrophobic | |
| dextrans: | |
| RG-94 | HM-Hb (80 kDa) equivalent to RG1193 |
| DAC | HM-Hb (80 kDa) |
| CR-27 | HM-Hb (80 kDa) |
| CR-29 | HM-Hb (400 kDa) |
| CR-31 | HM-Hb (1000 kDa) |
| CR-32 | HM-Hb (4000 kDa) |
| CR-36 | HM-Hb (20 kDa) |
| Non-sulfated dextrans: | |
| CR-34 | Intermediate of CR-36 without hydrophobic group |
| | and not sulfated. |
| CR-35 | Intermediate of CR-36 with hydrophobic |
| | group but not sulfated. |

*Inhibition with D120 tested at lower concentrations: 1 μg/ml = 40% inhibition, 0.5 μg/ml = 12% inhibition, 0.25 and 0.1 μg/ml = no inhibition.
Abbreviations:
HM-Hi (Heparan Mimetic hydrophilic)
HM-Hb (Heparan Mimetic hydrophilic/hydrophobic)
For general structures and synthesis, see Ledoux et al., 2000.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used, is intended to be in the nature of words of description rather than of limitation.

Obviously, many modifications and variations of the present invention are possible in light of the above teachings. It is, therefore, to be understood that within the scope of the described invention, the invention can be practiced otherwise than as specifically described.

### References

Ba-Charvet, K. T. N., Brose, K., Marillat, V., Kidd, T., Goodman, C. S., Tessier-Lavigne, M., Sotelo, C., and Chedotal, A. (1999) Slit2-mediated chemorepulsion and collapse of developing forebrain axons. Neuron 22:463-473.
Ba-Charvet, K. T N., Brose, K., Ma, L., Wang, K. H., Marillat, V., Sotelo, C., Tessier-Lavigne, M., and Chedotal, A. (2001) Diversity and specificity of actions of Slit2 proteolytic fragments in axon guidance. J. Neurosci. 21:4281-4289.
Ba-Charvet, K. T. N., Plump, A. S., Tessier-Lavigne, M., and Chedotal, A. (2002) Slit1 and slit2 proteins control the development of the lateral olfactory tract. J. Neurosci. 22:5473-5480.
Bandtlow, C. E. and Zimmermann, D. R. (2000) Proteoglycans in the developing brain: New conceptual insights for old proteins. Physiol. Rev. 80:1267-1290.
Barchi, J. J. Jr. (2000) Emerging roles of carbohydrates and glycomimetics in anticancer drug design. Curro Pharm. Des. 6:485-501.
Battye, R., Stevens, A., and Jacobs, J. R. (1999) Axon repulsion from the midline of the Drosophila CNS requires slit function. Development 126:2475-2481.
Benezra, M., Ishai-Michaeli, R., Ben-Sasson, S. A., and VIodavsky, I. (2002) Structure-activity relationships of heparin-mimicking compounds in induction of bFGF release from extracellular matrix and inhibition of smooth muscle cell proliferation and heparanaseactivity. J. Cellular Physiol. 192:276-285.
Bloechlinger, S., Karchewski, L. A., and Woolf, C. J., (2004) Dynamic changes in glypican-1 expression in dorsal root ganglion neurons after peripheral and central axonal injury, European Journal of Neuroscience, 19: 1119-1132.
Brose, K. and Tessier-Lavigne, M. (2000) Slit proteins: key regulators of axon guidance, axonal branching, and cell migration. Curro Opin. Neurobiol. 10:95-102.
Colamarino, S. A. and Tessier-Lavigne, M. (1995) The axonal chemoattractant netrin-1 is also a chemorepellent for trochlear motor axons. Cell 81 :621-629.
Compain, P. and Martin, O. R. (2001) Carbohydrate mimetics-based glycosyltransferase inhibitors. Bioorg. Med. Chem. 9:3077-3092.
Davis, A. P. and Wareham, R. S. (1999) Carbohydrate Recognition through Noncovalent Interactions: A Challenge for Biomimetic and Supramolecular Chemistry. Angew. Chem. Int. Ed. Engl. 38:2978-2996.
Dove, A. (2001) The bittersweet promise of glycobiology. Nat. Biotechnol. 19:913-917. Endo, T. (1999) O-mannosyl glycans in mammals. Biochem. Biophys. Acta 1473:237-246.
Englund, C., Steneberg, P., Falileeva, L., Xylourgidis, N., and Samakovlis, C. (2002) Attractive and repulsive functions of Slit are mediated by different receptors in the Drosophila trachea. Development 129:4941-4951.
Filmus, J., Church, J. G., and Bwick, R. N. (1988) Isolation of a cDNA corresponding to a developmentally regulated transcript in rat intestine. Mol. Cell. Biol. 8:4243-4249.
Finne, J., T. Krusius, R. K. Margolis, and R. U. Margolis. 1979. Novel mannitol-containing oligosaccharides obtained by mild alkaline borohydride treatment of a chondroitin sulfate proteoglycan from brain. J. Biol. Chem. 254:10295-10300.
Fraser, P. E., Darabie, A. A., and McLaurin, J. (2001) Amyloid-B interactions with chondroitin sulfate-derived. monosaccharides and disaccharides. Implications for drug development. J. Biol. Chem. 276:6412-6419.
Fromm, J. R., Hileman, R. E., Caldwell, E. E. O., Weiler, J. M., and Linhardt, R. J. (1995) Differences in the interaction of heparin with arginine and lysine and the importance of these basic amino acids in the binding of heparin to acidic fibroblast growth factor. Arch. Biochem. Biophys. 323:279-287.
Gadek, T. R. and Nicholas, J. B. (2003) Small molecule antagonists of proteins. Biochem. Pharmacol. 65:1-8. Giannattasio, G., A. Zanini, P. Rosa, J. Meldolesi, R. K. Margolis and R. U. Margolis 1980. Molecular organization of prolactin granules -III. Intracellular transport of glycosaminoglycans and glycoproteins of the bovine prolactin granule matrix. J. Cell Biol. 86:273-279.
Gagliardi, A. R. T., Kassack, M., Kreimeyer, A., Muller, G., Nickel, P., and Collins, D. C., (1998), Antiangiogenic and antiproliferative activity of suramin analogues, Cancer Chemother Pharmacol, 41: 117-124.
Gowda, D. C., B. Goossen, R. K. Margolis, and R. U. Margolis. (1989a) Chondroitin sulfate and heparan sulfate proteoglycans of PC12 pheochromocytoma cells. J. Biol. Chem. 26 4:11436-11443.
Gowda, D. C., Margolis, R. U., and Margolis, R. K. (1989b) Presence of the HNK-1 epitope on poly(N-acetyllactosaminyl) oligosaccharides and identification of multiple core proteins in the chondroitin sulfate proteoglycans of brain. Biochemistry 28:4468-4474.
Gowda, D. C., Hogue-Angeletti, R., Margolis, R. K., and Margolis, R. U. (1990) Chromaffin granule and PC12 cell chondroitin sulfate proteoglycans and their relation to chromogranin A. Arch. Biochem. Biophys. 281 :219-224.
Habuchi, H., Suzuki, S., Saito, T., Tamura, T., Harada, T., Yoshida, K., and Kimata, K. (1992) Structure of a heparan sulphate oligosaccharide that binds to basic fibroblast growth factor. Biochem. J. 285:805-813.
Hagino, S., Iseki, K., Mori, T., Zhang, Y., Hikake, T., Yokoya, S., Takeuchi, M., Hasimoto, H., Kikuchi, S., and Wanaka, A. (2003) Slit and glypican-1 mRNAs are coexpressed in the reactive astrocytes of the injured adult brain. Glia 42:130-138.
Hagino, S., K. Iseki, T. Mori, Y. Zhang, N. Sakai, S. Yokoya, T. Hikake, S. Kikuchi, A. Wanaka, (2003) Expression pattern of glypican-1 mRNA after brain injury in mice, Neurosci. Lett. 349: 29-32.
Hamann, M. C. J., E. C. Tsai, C. H. Tator, and M. S. Shoichet. (2003) Novel intrathecal delivery system for treatment of spinal cord injury. Experimental Neurology, 182: 300-309.
Hileman, R. E., Jennings, R. N., and Linhardt, R. J. (1998) Thermodynamic analysis of the heparin interaction with a basic cyclic peptide using isothermal titration calorimetry. Biochemistry 37:15231-15237.
Hu, H. (1999) Chemorepulsion of neuronal migration by Slit 2 in the developing mammalian forebrain. Neuron 23:703-711.
Hu, H. (2001) Cell-surface heparan sulfate is involved in the repulsive guidance activities of Slit2 protein Nature Neurosci. 4:695-701.
Iseki, K., Hagino, S., Mori, T., Zhang, Y., Yokoya, S., Takaki, H., Tase, C., Murakawa, M., and Wanaka, A. (2002) Increased syndecan expression by pleiotrophin and FGF receptor-expressing astrocytes in injured brain tissue. Glia 39:1-9.
Ishida, K., Wierzba, M. K., Teruya, T., Simizu, S., and Osada, H., (2004), Novel Heparan Sulfate Mimetic Compounds as Antitumor Agents, Chemistry & Biology, 11: 367-377.
Johnson, M. A. and Pinto, B. M. (2003) Molecular mimicry of carbohydrates by peptides. Aust J. Chem. 55:13-25.
Karthikeyan L., Maurel P., Rauch U., Margolis R. K., and Margolis R. U. (1992) Cloning of a major heparan—sulfate proteoglycan from brain and identification as the rat form of glypican. Biochem. Biophys. Res. Commun. 188,395-401.
Kiang, W.-L., C. P. Crockett, R. K. Margolis, and R. U. Margolis. 1978. Glycosaminoglycans and glycoproteins associated with microsomal subfractions of brain and liver. Biochemistry 17:3841-3848.
Kiang, W.-L., Margolis, R. U., and Margolis, R. K. (1981) Fractionation and properties of a chondroitin sulfate proteoglycan and the soluble glycoproteins of brain. J. Biol. Chem. 256:10529-10537.
Kiang, W.-L., T. Krusius, J. Finne, R. U. Margolis, and R. K. Margolis. 1982. Glycoproteins and proteoglycans of the chromaffin granule matrix. J. Biol. Chem., 257:1651-1659.
Kidd, T., Bland, K. S., and Goodman, C. S. (1999) Slit is the midline repellent for the robo receptor in Drosoghila. Cell 96:785-794.
Klinger, M. M., Margolis, R. U., and Margolis, R. K. (1985) Isolation and characterization of the heparan sulfate proteoglycans of brain. Use of affinity chromatography on lipoprotein lipase-agarose. J. Biol. Chem. 260:4082-4090.
Krusius, T., J. Finne, R. U. Margolis, and R. K. Margolis. 1978. Structural features of microsomal, synaptosomal, mitochondrial, and soluble glycoproteins of brain. Biochemistry 17:3849-3854.
Krusius, T., J. Finne, R. K. Margolis and R. U. Margolis. 1986. Identification of an O-glycosidic mannose-linked sialylated tetrasaccharide End keratan sulfate oligosaccharides in chondroitin sulfate proteoglycans of brain. J. Biol. Chem. 261 :8237-8242.
Krusius, T., V. N. Reinhold, R. K. Margolis, and R. U. Margolis. 1987. Structural studies on sialylated and sulfated a-glycosidic man nose-linked oligosaccharides in chondroitin sulfate proteoglycans of brain. Biochem. J. 245:229-234.
Ledoux, D., Merciris, D., Barritault, D., and Caruelle, J. P. (2003) Heparin-like dextran derivatives as well as glycosaminoglycans inhibit the enzymatic activity of human cathepsin G. FEBS Lett. 537:23-.29.
Ledoux, D., Merciris, D., Barritault, D., and Caruelle, J. P., Heparin-like dextran derivatives as well as glycosaminoglycans inhibit the enzymatic activity of human cathepsin G, FEBS Letters 537: 23-29.
Ledoux, D., Papy-Garcia, D., Escartin, Q., Sagot, M. A., Cao, Y., Barritault, D., Courtois, J., Hornebeck, W., and Caruelle, J. P., (2000), Human Plasmin Enzymatic Activity is Inhibited by Chemically Modified Dextrans, J. Biol. Chem. 275:29383-29390.
Li, H. S., Chen, J. H., Wu, W., Fagaly, T., Zhou, L., Yuan, W., DUPUIS, S., Jiang, Z. H., Nash, W., Gick, C., . Ornitz, D. M., Wu, J. Y., and Rao, Y. (1999) Vertebrate slit, a secreted ligand for the transmembrane protein roundabout, is a repellent for olfactory bulb axons. Cell 96:807-818.
Liang, Yu, Annan, R. S., Carr, S. A., Popp, S., Mevissen, M., Margolis, R. K., and Margolis, R. U., Mammalian Homologues of the Drosoghila Slit Protein are Ligands of the Heparan Sulfate Proteoglycan Glypican-1 in Brain, J. Biol. Chem. 274: 17885-17892.
Lindahl, U., Kusche-Gullberg, M., and Kjellen, L. (1998) Regulated diversity of heparan sulfate. J. Biol. Chem. 273:24979-24982.
Ma, a., Dudas, B., Daud, A., lqbal, O., Hoppensteadt, D., Jeske, W., Cornelli, U., Lee, J., Loren, S., Mervis, R., Hanin, I., Capila, I., Linhardt, R., and Fareed, J. (2002) Molecular and biochemical profiling of a heparin-derived oligosaccharide, C3. Thromb Res. 105:303-309.
Manetti, F., Cappello, V., Botta, M., Corelli, F., Mongelli, N., Biasoli, G., Borgia, A. L., and Ciomei, M., (1998) Synthesis and Binding Mode of Heterocyclic Analogues of Suramin Inhibiting the Human Basic Fibroblast Growth Factor, Bioorganic & Medicinal Chemistry, 6: 947-958.
Margolis, R. K., and R. U. Margolis. 1970. Sulfated glycopeptides from rat brain glycoproteins. Biochemistry 9:4389-4396.
Margolis, R. U., and R. K. Margolis. 1974. Distribution and metabolism of mucopolysaccharides and glycoproteins in neuronal perikaria, astrocytes, and oligodendroglia. Biochemistry 13:2849-2852.
Margolis R. U. and Margolis R. K. (Eds.) (1989) Neurobiology of Glycoconjugates, Plenum Publishing Corp.,” New York.
Margolis, R. K., R. U. Margolis, C. Preti, and D. Lai. (1975) Distribution and metabolism of glycoproteins and glycosaminoglycans in subcellular fractions of brain. Biochemistry 14:4797-4804.
Margolis, R. K., Thomas, M. D., Crockett, C. P., and Margolis, R. U. (1979) Presence of chondroitin sulfate in the neuronal cytoplasm. Proc. Natl. Acad. Sci. U. S. A. 76:1711-1715.
Margolis, R. K., Salton, S. R. J., and Margolis, R. U. (1983) Complex carbohydrates of cultured PC12 pheochromocytoma cells. Effects of nerve growth factor and comparison with neonatal and mature rat brain. J. Biol. Chem. 258:4110-4117.
Margolis, R. K., J. Finne, T. Krusius and R. U. Margolis. 1984. Structural studies on glycoprotein oligosaccharides of chromaffin granule membranes and dopamine flhydroxylase. Arch. Biochem. Biophys. 228:443-449.
Margolis, R. K., L. A. Greene, and R. U. Margolis. 1986. Poly(N-acetyllactosaminyl) oligosaccharides in glycoproteins of PC12 pheochromocytoma cells and sympathetic neurons. Biochemistry 25:3463-3468.
Margolis, R. K., B. Goossen, and R. U. Margolis. 1988a. Phosphatidylinositol-anchored glycoproteins of PC12 pheochromocytoma cells and brain. Biochemistry 27:3454-3458.
Margolis, R. U., R. Fischer-Colbrie, and R. K. Margolis. 1988b. Poly(N-acetyllactosaminyl) oligosaccharides of chromaffin granule membrane glycoproteins. J. Neurochem. 51:1819-1824.
Margolis, R. K., Goossen, B., Tekotte, H., Hilgenberg, L., and Margolis, R. U. (1991) Effects of B-xylosides on proteoglycan biosynthesis and morphology of PC12 pheochromocytoma cells and primary cultures of rat cerebellum. J. Cell Sci. 99:237-246.
Maurel, P., Meyer-Puttlitz, B., Flad, M., Margolis, R. U., and Margolis, R. K. (1995) Nucleotide sequence and molecular variants of rat receptor-type protein tyrosine phosphatase-′(jj3. DNA Sequence 5:323-328.
Michele, D. E., Barresi, R., Kanagawa, M., Saito, F., Cohn, R. D., Satz, J. S., Dollar, J., Nishino, I., Kelley, R. I., Somer, H., Straub, V., Mathews, K. D., Moore, S. A., and Campbell, K. P. (2002) Post-translational disruption of dystroglycan-ligand interactions in congenital muscular dystrophies. Nature 418:417-422.
Melo, F. R., Pereira, M. S., Foguel, D., and Mourao, P. A. S., (2004) Antithrombin-mediated Anticoagulant Activity of Sulfated Polysaccharides, J. Biol. Chem. 279: 20824-20835.
Moore, S. A., Saito, F., Chen, J., Michele, D. E., Henry, M. D., Messing, A., Cohn, R. D., Ross-Barta, S. E., Westra, S., Williamson, R. A., Hoshi, T., and Campbell, K. P. (2002) Deletion of brain dystroglycan recapitulates aspects of congenital muscular dystrophy. Nature 418:422-425.
Nakato, H., Futch, T. A., and Selleck, S. B. (1995) The division abnormally delayed (dally) gene: a putative integral membrane proteoglycan required for cell division patterning during postembryonic development of the nervous system in Drosoghila. Development 121 :3687-3702.
Ornitz, D. M. (2000) FGFs, heparan sulfate and FGFRs: complex interactions essential for development. BioEssays 22:108-112.
Park, P. W., Reizes, O., and Bernfield, M. (2000) Cell surface heparan sulfate proteoglycans: selective regulators of ligand-receptor encounters. J. Biol. Chem. 275:29923-29926.
Perrimon, N., and Bernfield, M. (2000) Specificities of heparan sulphate proteoglycans in developmental processes. Nature 404:725-728.
Pervin, A., Gallo, C., Jandik, K. A., Han, X.-J., and Linhardt, R. J. (1995) Preparation and structural characterization of large heparin-derived oligosaccharides. Glycobiology 5:83-95.
Petitou, M., Herault, J. P., Bernat, A., Driguez, P. A., Duchaussoy, P., Lormeau, J. C., and Herbert, J. M. (1999) Synthesis of thrombin-inhibiting heparin mimetics without side effects. Nature 398:41.7-422.
Pilia, G., Hughes-Benzie, R. M., MacKenzie, A., Baybayan, P., Chen, E. Y., Huber, R., Neri, G., Cao, A., Forabosco, A., and Schlessinger, D. (1996) Mutations in GPC3, a glypican gene, cause the Simpson- Golabi-Behmel overgrowth syndrome. Nature Genetics 12:225-227.
Pinhal, M. A., Trindade, E. S., Fareed, J., Dietrich, C. P., and Nader, H. B. (2001) Heparin and a cyclic octaphenol-octasulfonic acid (GL-522-Y-1) bind with high affinity to a 47-kda protein from vascular endothelial cell surface and stimulate the synthesis and structural changes of heparan sulfate proteoglycan. Thromb Res. 103:35-45.
Piper, M. and Little, M. (2002) Movement through Slits: cellular migration via the Slit family. BioEssays 25:32-38.
Prieto, A. L., Andersson-Fisone, C., and Crossin, K. L. (1992) Characterization of multiple adhesive and counteradhesive domains in the extracellular matrix protein cytotactin. J. Cell Biol. 119:663-678.
Qing, M. Dudas, B., Daud, A. lqbal, O., Hoppensteadt, D., Jeske, W., Cornelli, U., Lee, J., Lorens, S., Mervis, R., Hanin, I., Capila, I., Linhardt, R., and Fareed, J., Molecular and biochemical profiling of a heparin-derived oligosaccharide, C3, Thrombosis Research, 105: 303-309.
Randell, K. D., Barkley, A., and Arya, P. (2002) High-throughput chemistry toward complex carbohydrates and carbohydrate-like compounds. Comb. Chem High Throughput Screen. 5: 17˜-193.
Rauch, U., Gao, P., Janetzko, A., Flaccus, A., Hilgenberg, L., Tekotte, H., Margolis, R. K., and Margolis, R. U. (1991) Isolation and characterization of developmentally-regulated chondroitin sulfate and chondroitin/keratan sulfate proteoglycans of brain identified with monoclonal antibodies. J. Biol Chem. 266:14785-14801.
Rauch, U., L. Karthikeyan, P. Maurel, R. U. Margolis, and R. K. Margolis. (1992) Cloning, and primary structure of neurocan, a developmentally regulated, aggregating chondroitin sulfate proteoglycan of brain. J. Biol. Chem. 267: 19536-19547.
Richards, L. J. (2002) Surrounded by Slit—how forebrain commissural axons can be led astray. Neuron 33:153-158.
Ripellino, J. A., Klinger, M. M., Margolis, R. U., and Margolis, R. K. (1985) The hyaluronic acid binding region as a specific probe for the localization of hyaluronic acid in tissue sections. Application to chick embryo and rat brain. J. Histochem. Cytochem. 33:1060-1066.
Ripellino, J. A., Bailo, M., Margolis, R. U., and Margolis, R. K. (1988) Light and electron microscopic studies on the localization of hyaluronic acid in developing rat cerebellum. J. Cell Biol. 106:845-855.
Ripellino, J. A. and Margolis, R. U. (1989) Structural properties of the heparan sulfate proteoglycans of brain. J. Neurochem. 52:807-812.
Ronca, F, Andersen, J. S., Paech, V., and Margolis, R. U. (2001) Characterization of Slit Protein Interactions with Glypican-1. J. Biol. Chem. 276:29141-29147.
Rothberg, J. M., Jacobs, J. R., Goodman, C. S., and Artavanis-Tsakonas, S. (1990) Slit: An extracellular protein necessary for development of midline glia and commissural axon pathways contains both EGF and LRR domains. Genes Dev. 4:2169-2187.
Salton, S. R. J., Margolis, R. U., and Margolis, R. K. (1983) Release of chromaffin granule glycoproteins and proteoglycans from potassium-stimulated PC12 pheochromocytoma cells. J. Neurochem. 41: 1165-1170.
Sang, Q., Wu, J., Rao, Y., Hsueh, Y, Po, and Tan, S. S. (2002) Slit promotes branching and elongation of neurites of interneurons but not projection neurons from the developing telencephalon. Mol. Cell. Neurosci. 21 :250-265.
Schlessinger, J., Lax, I., and Lemmon, M. (1995) Regulation of growth factor activation by proteoglycans: What is the role of the low affinity receptors? Cell 83:357-360.
Sears, P. and Wong, C. H. (1999) Carbohydrate Mimetics: A New Strategy for Tackling the Problem of Carbohydrate-Mediated Biological Recognition. Angew. Chem. Int Ed. Engl. 38:2300-2324.
St Hilaire, P. M. and Meldal, M. (2000) Glycopeptide and Oligosaccharide Libraries. Angew. Chem. Int Ed. Engl. 39:1162-1179.
Tang, X., Davies, J. E., and Davies, J. A. (2003) Changes in distribution, cell associations, and protein expression levels of NG2, neurocan, phosphacan, brevican, versican V2, and tenascin-C during acute to chronic maturation of spinal cord scar tissue. J. Neurosci. Res. 71 :427-444.
Tardieu, M., Gamby, C., Avramoglou, T., Jozefonvicz, J., and Barritault, D. (1992) Derivatized dextrans mimic heparin as stabilizers, potentiators, and protectors of acidic or basic FGF. J. Cellular Physiol. 150:194-203.
Tumova, S., Woods, A., and Couchman, J. R. (2000) Heparan sulfate proteoglycans on the cell surface: versatile coordinators of cellular functions. IJBCB 32:269-288.
Turnbull, J. E., Fernig, D. G., Ke, Y., Wilkinson, M. C., and Gallagher, J. T. (1992) Identification of the basic fibroblast growth factor binTjing sequence in fibroblast heparan sulfate. J. Biol. Chem. 267: 10337-10341.
Turnbull, J., Powell, A., and Guimond, S. (2001) Heparan sulfate: decoding a dynamic multifunctional cell regulator. Trends Cell Biol. 11 :75-82.
Wu, W., Wong, K., Chen, J.-h., Jiang, Z.-h., Dupuis, S., Wu, J. Y., and Rao, Y. (1999) Directional guidance of neuronal migration in the olfactory system by the protein Slit. Nature 400:331.-3˜6.
Yamaguchi, Y. (2001) Heparan sulfate proteoglycans in the nervous system: their diverse roles in neurogenesis, axon guidance, and synaptogenesis. Cell & Dev. Biol. 12:99-106.
Yoshida, A., Kobayashi, K., Manya, H., Taniguchi, K., Kano, H., Mizuno, M., Inazu, T., Mltsuhashl, H., Takahashi, S., Takeuchi, M., Herrmann, R., Straub, V., Talim, B., Voit, T., Topaloglu, Toda, T., and Endo, T. (2001) Muscular dystrophy and neuronal migration disorder caused by mutations In a glycosyltransferase, POMGnT1. Dev. Cell 1:717-724.
Yu, G., Gunay, N. S.; Linhardt, R. J., Toida, T., Fareed, J., Hoppensteadt, D. A., Shadid, H., Ferro, V., II, C., Fewings, K., Palermo, M. C., and Rodger, D. (2002) Preparation and anticoagulant activity of the phosphosulfomannan PI-88. Eur. J. Med. Chem. 37:783-791.
Zanini, A., G. Giannattasio, G. Nussdorfer, R. K. Margolis, R. U. Margolis, and J. Meldolesi. 1980. Molecular organization of prolactin granules -II. Characterization of glycosaminoglycans and glycoproteins of the bovine prolactin granule matrix. J. Cell Biol. 86:260-272.
Zhang, F., M. Fath, R. Marks, R. J. Linhardt, (2002) A highly stable covalent conjugated heparin biochip for heparin-protein interaction studies, Anal. Biochem. 304 (2002) 271-273.
Zhang, J., Rivers, G., Yanyi, Z., Jacobson, A., Peyers, J., Grundstrom, G., Burch, P., Hussein, S., Marolewski, A., Herlihy, W., and Rusche, J, (2001) Identification of Inhibitors of Heparin-Growth Factor Interactions from Combinatorial Libraries of Four-Component Condensation Reactions. Bioorganic & Medicinal Chemistry, 9: 825-836.
Zhang, F., F. Ronca, R. J. Linhardt, and R. U. Margolis, (2004), Structural Determinants of heparin sulfate interactions with Slit proteins, Biochemical and Biophysical Research Communications. 317: 352-357.

1. A composition for inhibiting slit protein and glypican interactions comprising an effective amount of a heparin mimetic.

2. The composition according to claim 1, wherein said heparin mimetic is a compound selected from the group consisting of chemically modified glycosaminoglycans such as hyaluronic acid, dermatan sulfate, chondroitin sulfate, heparin, heparan sulfate, and keratan sulfate, low molecule weight heparin-mimetic compounds, heparin oligosaccharides, heparin-like glycosaminoglycans (HLGAGs), suramin, suramin-like compounds, polyanions such as dextran sulfate, sulfated polysaccharides, negatively charged serum albumin and milk proteins, synthetic sulfated polymers, polymerized anionic surfactants and polyphosphates, various sulfated molecules, various sulfonated molecules, synthetic polyaromatic compounds, polyaromatic compounds synthesized by polymerization of aromatic ring monomers with formaldehyde, polysulfated dyes, Reactive Black 5, Remazol Brilliant Blue R, Reactive Orange 16, trypan blue, α-cyclodextrin sulfate, fully sulfated maltotrioside prepared as a precursor for synthetic heparins, water-soluble synthetic dextran derivatives such as those containing sulfate, carboxymethyl, and benzylamide groups on the OH residues of glucose units, randomly derivatized dextrans, sulfated phosphomannan, heparin-derived oligosaccharide C3, a cyclic octaphenol-octasulfonic acid, a low-molecular weight fragment of heparin prepared by chemical or enzymatic depolymerization, fragments thereof, and combinations thereof.

3. The composition according to claim 2, further comprising a compound selected from the group consisting of platelet factor IV, prothrombin, vitamin K, fibrinogen, prothrombin, thromboplastin, tissue factor, calcium, labile factor, stable factor, antihemophilic globulin (AHF), antihemophilic globulin (AHG), antihemophilic factor A, plasma thromboplastin component, Christmas factor, antihemophilic factor B, Stuart factor, Prower factor, Stuart-Prower factor, plasma thromboplastin antecedent (PTA), antihemophilic factor C Hageman factor, surface factor, contact factor, fibrin stabilizing factor (FSF), fibrin stabilizing enzyme, fibrinase, prekallikrein (Fletcher factor), high molecular weight kininogen , blood clotting or coagulation factors, and combinations thereof.

4. A pharmaceutical composition for inhibiting slit protein and glypican interactions comprising the composition according to claim 1 and a pharmaceutical carrier.

5. A composition for promoting axonal regeneration comprising an effective amount of a heparin mimetic.

6. The composition according to claim 5, wherein said heparin mimetic is a compound selected from the group consisting of chemically modified glycosaminoglycans such as hyaluronic acid, dermatan sulfate, chondroitin sulfate, heparin, heparan sulfate, and keratan sulfate, low molecule weight heparin-mimetic compounds, heparin oligosaccharides, heparin-like glycosaminoglycans (HLGAGs), suramin, suramin-like compounds, polyanions such as dextran sulfate, sulfated polysaccharides, negatively charged serum albumin and milk proteins, synthetic sulfated polymers, polymerized anionic surfactants and polyphosphates, various sulfated molecules, various sulfonated molecules, synthetic polyaromatic compounds, polyaromatic compounds synthesized by polymerization of aromatic ring monomers with formaldehyde, polysulfated dyes, Reactive Black 5, Remazol Brilliant Blue R, Reactive Orange 16, trypan blue, α-cyclodextrin sulfate, fully sulfated maltotrioside prepared as a precursor for synthetic heparins, water-soluble synthetic dextran derivatives such as those containing sulfate, carboxymethyl, and benzylamide groups on the OH residues of glucose units, randomly derivatized dextrans, sulfated phosphomannan, heparin-derived oligosaccharide C3, a cyclic octaphenol-octasulfonic acid, a low-molecular weight fragment of heparin prepared by chemical or enzymatic depolymerization, fragments thereof, and combinations thereof.

7. The composition according to claim 6, further comprising a compound selected from the group consisting of platelet factor IV, prothrombin, vitamin K, fibrinogen, prothrombin, thromboplastin, tissue factor, calcium, labile factor, stable factor, antihemophilic globulin (AHF), antihemophilic globulin (AHG), antihemophilic factor A, plasma thromboplastin component, Christmas factor, antihemophilic factor B, Stuart factor, Prower factor, Stuart-Prower factor, plasma thromboplastin antecedent (PTA), antihemophilic factor C Hageman factor, surface factor, contact factor, fibrin stabilizing factor (FSF), fibrin stabilizing enzyme, fibrinase, prekallikrein (Fletcher factor), high molecular weight kininogen , blood clotting or coagulation factors, and combinations thereof.

8. A method for inhibiting slit protein and glypican interaction comprising the steps of administering the composition according to claim 1.

9. A method for promoting axonal regeneration comprising the steps of administering the composition according to claim 5.

10. A method for treating spinal cord injury comprising the steps of administering the composition according to claim 1.

11. A therapeutic composition for inhibiting slit protein and glypican interaction comprising an effective amount of a heparin mimetic.

12. The therapeutic composition according to claim 11, wherein said heparin mimetic is a compound selected from the group consisting of chemically modified glycosaminoglycans such as hyaluronic acid, dermatan sulfate, chondroitin sulfate, heparin, heparan sulfate, and keratan sulfate, low molecule weight heparin-mimetic compounds, heparin oligosaccharides, heparin-like glycosaminoglycans (HLGAGs), suramin, suramin-like compounds, polyanions such as dextran sulfate, sulfated polysaccharides, negatively charged serum albumin and milk proteins, synthetic sulfated polymers, polymerized anionic surfactants and polyphosphates, various sulfated molecules, various sulfonated molecules, synthetic polyaromatic compounds, polyaromatic compounds synthesized by polymerization of aromatic ring monomers with formaldehyde, polysulfated dyes, Reactive Black 5, Remazol Brilliant Blue R, Reactive Orange 16, trypan blue, α-cyclodextrin sulfate, fully sulfated maltotrioside prepared as a precursor for synthetic heparins, water-soluble synthetic dextran derivatives such as those containing sulfate, carboxymethyl, and benzylamide groups on the OH residues of glucose units, randomly derivatized dextrans, sulfated phosphomannan, heparin-derived oligosaccharide C3, a cyclic octaphenol-octasulfonic acid, a low-molecular weight fragment of heparin prepared by chemical or enzymatic depolymerization, fragments thereof, and combinations thereof.

13. The composition according to claim 12, further comprising a compound selected from the group consisting of platelet factor IV, prothrombin, vitamin K, fibrinogen, prothrombin, thromboplastin, tissue factor, calcium, labile factor, stable factor, antihemophilic globulin (AHF), antihemophilic globulin (AHG), antihemophilic factor A, plasma thromboplastin component, Christmas factor, antihemophilic factor B, Stuart factor, Prower factor, Stuart-Prower factor, plasma thromboplastin antecedent (PTA), antihemophilic factor C Hageman factor, surface factor, contact factor, fibrin stabilizing factor (FSF), fibrin stabilizing enzyme, fibrinase, prekallikrein (Fletcher factor), high molecular weight kininogen, blood clotting or coagulation factors, and combinations thereof.

14. A therapeutic composition for promoting axonal regeneration comprising an effective amount of a heparin mimetic.

15. The therapeutic composition according to claim 14, wherein said heparin mimetic is a compound selected from the group consisting of chemically modified glycosaminoglycans such as hyaluronic acid, dermatan sulfate, chondroitin sulfate, heparin, heparan sulfate, and keratan sulfate, low molecule weight heparin-mimetic compounds, heparin oligosaccharides, heparin-like glycosaminoglycans (HLGAGs), suramin, suramin-like compounds, polyanions such as dextran sulfate, sulfated polysaccharides, negatively charged serum albumin and milk proteins, synthetic sulfated polymers, polymerized anionic surfactants and polyphosphates, various sulfated molecules, various sulfonated molecules, synthetic polyaromatic compounds, polyaromatic compounds synthesized by polymerization of aromatic ring monomers with formaldehyde, polysulfated dyes, Reactive Black 5, Remazol Brilliant Blue R, Reactive Orange 16, trypan blue, α-cyclodextrin sulfate, fully sulfated maltotrioside prepared as a precursor for synthetic heparins, water-soluble synthetic dextran derivatives such as those containing sulfate, carboxymethyl, and benzylamide groups on the OH residues of glucose units, randomly derivatized dextrans, sulfated phosphomannan, heparin-derived oligosaccharide C3, a cyclic octaphenol-octasulfonic acid, a low-molecular weight fragment of heparin prepared by chemical or enzymatic depolymerization, fragments thereof, and combinations thereof.

16. The therapeutic composition according to claim 15, further comprising a compound selected from the group consisting of platelet factor IV, prothrombin, vitamin K, fibrinogen, prothrombin, thromboplastin, tissue factor, calcium, labile factor, stable factor, antihemophilic globulin (AHF), antihemophilic globulin (AHG), antihemophilic factor A, plasma thromboplastin component, Christmas factor, antihemophilic factor B, Stuart factor, Prower factor, Stuart-Prower factor, plasma thromboplastin antecedent (PTA), antihemophilic factor C Hageman factor, surface factor, contact factor, fibrin stabilizing factor (FSF), fibrin stabilizing enzyme, fibrinase, prekallikrein (Fletcher factor), high molecular weight kininogen , blood clotting or coagulation factors, and combinations thereof.
